(19) United States
(12) Patent Application Publication
(10) Pub. No.: US 2024/0360487 A1
(43) Pub. Date: Oct. 31, 2024

(54) A COMPOSITION FOR ANTI-BACTERIAL BIO-CELLULOSIC PATCHES USEFUL FOR TRANSDERMAL DRUG DELIVERY AND A PROCESS FOR THE PREPARATION THEREOF

(71) Applicant: COUNCIL OF SCIENTIFIC AND INDUSTRIAL RESEARCH AN INDIAN REGISTERED BODY INCORPORATED UNDER THE REGN., New Delhi (IN)

(72) Inventors: Saurabh SARAN, Jammu Jammu Kashmir (IN); Manoj KUMAR, Jammu Jammu Kashmir (IN); Shifali CHIB, Jammu Jammu Kashmir (IN); Rahul BHAT, Jammu Jammu Kashmir (IN); Utpal NANDI, Jammu Jammu Kashmir (IN); Ashish DOGRA, Jammu Jammu Kashmir (IN); Inshad Ali KHAN, Jammu Jammu Kashmir (IN)

(21) Appl. No.: 18/691,945

(22) PCT Filed: Sep. 30, 2022
(86) PCT No.: PCT/IN2022/050876

§ 371 Date: Mar. 14, 2024

(30) Foreign Application Priority Data
Oct. 1, 2021 (IN) 202111044817

(51) Int. Cl. C12P 19/04; A61K 9/70; C12N 1/20; C12R 1/01

(52) U.S. Cl. CPC C12P 19/04; A61K 9/703; C12N 1/205; C12N 2511/00; C12R 2001/01

(57) ABSTRACT

The present invention relates to the development of antibacterial biocellulosic patches/membranes for transdermal drug delivery. Novel high cellulose producing bacterial strain Komagatacibacter hansenii (MBS-8) has been isolated. The production and purification process of biocellulosic membranes is developed in such a way that the membranes produced are of desired thickness and length, suitable for drug impregnation in a cost-effective manner for the development of transdermal patches. Further, a process for efficient drug impregnation of Mupirocin in BC membranes has been developed. Weight and drug content of mupirocin impregnated BCM were found to be uniform.

### FIELD OF THE INVENTION

The present invention relates to a composition for anti-bacterial bio-cellulosic patches/membranes useful for transdermal drug delivery and a process for the preparation thereof. The invention further relates to a method for cost effective production of bacterial cellulose membranes for transdermal drug delivery. More particularly, the instant invention relates to the isolation of a bacterial cellulose producer, optimization, impregnation and delivering of topical antibiotic including Mupirocin using bio-cellulosic patches.

Bacterial cellulose has been used for drug loading and controlled release by making transdermal patches. Transdermal patches comprise a method of delivering medication through the skin in a non-invasive manner. The patch is designed in such a way that the medication permeates the skin in a controlled fashion thus attaining more steady levels of the drug in the body and helps in wound healing. The membranes developed in the present invention find immense application in the healthcare sector, especially for the treatment of dermal wound infections using medicated bacterial cellulosic patches in a non-invasive manner.

### BACKGROUND OF THE INVENTION

Bacterial cellulose (BC) has the same molecular structure as that of plant cellulose. However, bacterial cellulose exhibits wide range of properties and is renowned for its high crystallinity index, high degree of polymerization, high tensile strength, and water holding capacity, purity, and biodegradability. It has high aspect ratio with a diameter of 20-100 nm. As an outcome, BC has a very high surface area per unit mass. This feature, associating with its highly hydrophilic nature, results in a very high liquid loading capacity. Further, the biocompatibility of BC, hydrophilicity, biocompatibility, transparency, and non-toxicity make it an interesting polymer for a wide range of applications in various fields.

Organisms that produce microbial cellulose include Algae (Valonia), fungi (Saprolegnia, Dictyostelium discoideum), bacteria (Gluconacetobacter, Achromobacter, Azotobacter, Aerobacter, Agrobacterium, Pseudomonas, Rhizobium, Sarcina, Alcaligenes, Zoogloea). The most familiar cellulose producing bacteria are members of the family Acetobactereaceae and belong to the genera Komagataeibacter (formerly Acetobacter and currently Gluconacetobacter genus).

Transdermal drug delivery is an exciting and challenging area. There are few transdermal delivery systems currently available in the market. However, the transdermal market still remains limited to a narrow range of drugs. Further advances in transdermal delivery systems based on the economical production of bacterial cellulose membrane using microorganisms and the unique properties of BC will overcome the challenges faced regarding the permeation and drug release. These transdermal patches offer many benefits over other route of drug administration.

By using medicated patches, direct-to-bloodstream delivery while bypassing the liver's metabolic activity is possible. The patient's body heat activates a patch, prompting it to begin releasing medication through the skin into the bloodstream. Secondly, using these patches, the medication is supplied gradually and constantly, rather than in a large, single dose. Patches utilize the skin's natural barrier properties to achieve a constant permeation of the drug and achieve steadier blood levels as compared to injectables and most oral medications. Another benefit of using patches is that their use allows a medication to bypass the acidic/alkaline environment found in the digestive system. Additionally, patches are painless, eliminates the need for injections that can cause a patient irritation and discomfort. Patches are simply placed on the skin, worn for a prescribed period of time, and removed. This also makes them convenient.

Scientists have not attempted much on the research for the production of bacterial cellulose and its applications. A survey of literature shows that there are a few publications that directly address the productions and applications of microbial cellulose. However, no research has been carried out on the development of transdermal patches using BC membranes.

Reference may be made to Pavaloiu R D, et al. 2014 (DOI: 10.2478/s11532-014-0541-3) which reports the study on the release of amoxicillin from bacterial cellulose membranes but in this article no study was done in-vivo, and the drug used is not mupirocin.

Reference may be made to U.S. Pat. No. 9,314,531B2, which recites a wound healing composition comprising a biocompatible hydrogel membrane and provides methods of treating a wound in a subject in need thereof. However, the drawbacks associated with the said document are of no use of any wound infected pathogen and no in-vivo studies were made in this work.

Reference may be made to CN107177048A, which recites a kind of bacterial cellulose polymeric lactic acid compound film and preparation method thereof to load medicine gauze and preparation method based on the composite membrane. However, the drawbacks associated with the said document are that they only concentrated on the preparation of medicine gauze for loading of drug, neglecting the study on animal model.

Reference may be made to CN105797197B, which recites a kind of dressing for skin and preparation method using bacteria cellulose as conductive surface substrate. The conductive surface is used for on-load voltage, to control the rate of release of the drug on skin contact surface, while for simulating biological endogenous electric field. However, the drawbacks associated with the said document are the lack of use of any in-vivo study and they merely concentrated on the preparation of dressing, lacking any work using skin pathogens.

A comparison of the relevant prior arts highlighting their findings and drawbacks vis-à-vis the present invention is summarized in the Table below:

| | | | Drawbacks vis-à-vis |
|---|---|---|---|
| Prior Art | Microorganism | Application | the present invention |
| Hongyang et al. | Bacillus | Cellulose based wet | Lacks any |
| 2018. A kind of | amyloliquefaciens | antibacterial dressing | antibacterial study |
| moist antiseptic | ZF-7 | having polylysine for | elaborating role of |
| dressing of bacteria | | chronic non-healing | dressing against |
| cellulose-base and | | wounds, subacute | pathogens. |
| preparation method | | refractory wounds, | |
| | | burns and the | |
| | | treatment of large-area | |
| | | soft tissue defects | |
| | | and/or infections. | |
| Xi Tingfei et al. | | The patch has | Recites only the |
| 2014Bacterial | | excellent mechanical | preparation of |
| cellulose biological | | property, better | patch lacking any |
| patch | | biological fitness, | antibacterial |
| &manufacturing | | better anti-adhesion | component along |
| method | | property | with membranes. |
| Heather Beam et | Acetobacter | Implantable microbial | Lacking any |
| al. 2006 Implantable | xylinum | cellulose for tissue | antibacterial study |
| microbial cellulose | | closure reinforcement, | against pathogens. |
| materials for | | buttresses for | |
| various medical | | reinforcement of the | |
| applications | | soft tissue, rotator cuff | |
| | | repair, and as carrier | |
| | | vehicles for medically | |
| | | useful substances for | |
| | | repair or regeneration | |
| | | of tissue. | |
| Zhijiang et al. | Gluconacetobacter | Chitosan/carboxymethyl | Composite lacks |
| 2017A kind of | xylinus | chitosan-based BC and | study in animal |
| preparation method | TJU S8 | against Escherichia coli | model |
| of antibacterial | | and staphylococcus aureus | |
| composite bacterial | | growth | |
| cellulose film | | | |
| Palomar et al., 2017 | Komagataeibacter | Produced bacterial | Only preparation |
| Method for | xylinus | cellulose may be used | of cellulose. No |
| producing bacterial | strain DSMZ | for biomedical | study to show the |
| cellulose | | application | use of the same as |
| membranes useful | | | antimicrobial |
| in biomedical | | | patch. |
| applications | | | |
| Jin et a.l, 2015 | Gluconacetobacter | Antimicrobial peptide | Antibacterial |
| Preparation method | GD-BC-1 | produced by bacteria | bacterial cellulose |
| of bacterial | | | membranes. Lack |
| cellulose with | | | any study against |
| antimicrobial | | | pathogens and in |
| property | | | any animal model. |
| Present Invention | Komagataeibacter | Production of bacterial | |
| | hansenii | cellulose and | |
| | (MTCC 13036) | transdermal drug | |
| | | delivery of mupirocin | |
| | | impregnated bacterial | |
| | | cellulose in BALB/c | |
| | | MICE infected with S. | |
| | | aureus MRSA 15187 | |

A review of the prior art reveals that the bacterial cellulose obtained from Gluconacetobacter or Acetobacter have been manufactured as dressing, hydrogel membrane, film, bandage, plaster, artificial skin, etc. to treat skin wounds, injuries, burns, surgery infections and repair of muscles, skin tissues. However, there is no report issued on isolating a potent cellulose producer which can produce bacterial cellulose in high quantities and a composition for anti-bacterial bio-cellulosic patches comprising bacterial cellulose which when mixed with other ingredients and antibiotics result in a composition that is suitable for transdermal drug delivery.

Thus, keeping in view the drawbacks of the Hitherto reported prior art the inventors of the present invention realized that there exist a dire need to provide a composition for anti-bacterial bio-cellulosic patches comprising bacterial cellulose membrane impregnated with antibiotics and other ingredients such that the resulting product can be used for transdermal drug delivery. The present invention aims to avert the shortcomings of the prior art by providing a composition for anti-bacterial bio-cellulosic patches having mupirocin incorporated therein for use as topical antibiotic to allow a slow release of the antibiotic on the wound infected site; further taking glycerol as plasticizer to provide flexibility to the patches. Such kind of mupirocin incorporated BC membranes have not been reported till date.

### Objectives of the Invention

The main objective of the present invention is therefore to provide a composition for anti-bacterial bio-cellulosic patches useful for transdermal drug delivery which obviates the drawbacks of the Hitherto reported prior art.

Another objective of the present invention is to provide a method for cost effective production of bacterial cellulose membranes and antibacterial bio-cellulosic patches for transdermal drug delivery.

Yet another objective of the present invention is to provide antibacterial bio-cellulosic patches comprising the antibiotic mupirocin for transdermal drug delivery.

Still another objective of the present invention is to provide an isolated bacterial strain of Komagataeibacter hansenii capable of producing bacterial cellulose membrane useful for transdermal drug delivery.

Yet another object of the present invention is to provide in-vivo and in-vitro drug release study and permeation study of the drug impregnated BC patches.

### SUMMARY OF THE INVENTION

Bacterial cellulose itself does not have antibacterial properties. The present disclosure there is provided a composition for Bacterial Cellulose [BC] membranes in the form of patches having a definite amount of antibiotic mupirocin along with other ingredients which gets released form membrane when applied on wound infections.

The bacterial cellulose producer of the present invention was isolated from rotten apple having small circular rough colonies were found to be Gram-negative bacteria. Initially the bacteria were grown in Hestrin schramm medium having 2% Glucose, Peptone 0.5%, Yeast 0.5%, Disodium hydrogen phosphate-0.27%, Citric acid-0.115%. pH=7, Temperature—30° C., under static culture.

For optimization of the medium composition resulting in highest bacterial cellulose production, different medium and parameters were considered for maximum production of bacterial cellulose. Production and growth requirement after optimization-Glycerol 2-4%, glucose—0.5%-2%, Peptone—0.5%-1%, Disodium hydrogen phosphate—0.27%-0.50%, Citric acid—0.015%-0.025%, Temperature—28-30° C., pH=5.5 to 6.2, Incubation time 7-9 days, inoculum age 4-6 days, Size of inoculum 1.5%-2% for clinical and drug delivery studies BC membranes were washed with weak alkali and weak acid and autoclaved at 121.5° C. for 15 minutes. Transdermal patches of bacterial cellulose with 2.5% & 5.0% loading of topical antibiotic mupirocin was successfully developed. Drug content study showed mupirocin content 590±19 μg per patch.

The next aim was to determine the potential effectiveness of application of BC impregnated mupirocin patch at the site of infected dermal area. Combination study of mupirocin with the novel drug delivery natural polymer i.e., bio cellulose membrane inhibited bacterial growth in-vivo. Incorporation of mupirocin in BC membrane exhibited 0.5 to 2.5 fold log CFU bacterial reduction with varying concentration from 50 to 500 μg/patch after single application.

The developed composition [interchangeably used as formulation] with single time application exhibited good efficacy in-vivo as compared to 2% mupirocin alone. Mupirocin 2% with one time application exhibited 0.5-fold log CFU reduction on 1^{st} day, but on 5th day CFU count of infected site eventually increased to 4.0×10⁶. No such log CFU difference was distinguished between control and Placebo treated groups at the end of the day of experiment. Pharmacokinetic study for mupirocin impregnated BC membrane was carried out to obtain the mean plasma concentration of mupirocin at different point of time and mupirocin was detected in plasma. Acute dermal toxicity of mupirocin impregnated BC membrane was investigated in comparison to blank BC membrane to identify any toxic effect. There were no notable clinical signs of toxicity and mortality observed in the experimental animals. Overall body weight of animals increased with respect to time in both the groups. Though no marked changes were observed in the relative organ weight yet there were some changes observed in hematological parameters and biochemical parameters. Mupirocin holds the potential of BC membrane and efficacy of one-time application on infected wounds was analyzed in the present study. The developed antibacterial bio-cellulosic patches were found effective in reducing the CFU count of the infected skin patch than exhibited by commercially available mupirocin (T-Bact) alone.

In an embodiment of the present disclosure bacterial cellulose patches were produced by growing Komagataeibacter hansenii in small trays in static aerobic conditions medium having glucose 0.5%-2%, glycerol, 2%-4% as carbon source, peptone 0.5%-1%, yeast extract 0.25%-1% as nitrogen source and disodium hydrogen phosphate 0.27%-0.50%, citric acid 0.015%-0.025% as salts, 1.5%-2% inoculum concentration, 4-6 days inoculum age, 7-9 days incubation time at pH ranging from 5.5 to 6.2 and temperature ranging from 28-30° C.

In another embodiment cellulose production was carried out in 250 ml trays in medium having alternate carbon source (glucose and glycerol) and an economic process was developed to wash produced bacterial cellulose in which harvested bacterial cellulose was washed with boiled 1 N NaOH for 1 hour and then dipped in 0.8N glacial acetic acid for 4 hours and then washed with distilled water until the pH of water comes out to be neutral.

In yet another embodiment mupirocin impregnated bacterial cellulose was characterized by different techniques namely Weight variation test, Drug content assay, Differential Scanning calorimetry, Thermal Gravimetric Analysis, Fourier-Transform Infrared Spectroscopy, Scanning Electron Microscopy for confirming and quantifying successful impregnation in the developed BC patches.

In still another embodiment permeation, Pharmacokinetic study were done to obtain the mean plasma concentration of mupirocin at different point of to confirm the release of mupirocin and to study in-vivo efficacy for assessing the role of bacterial cellulose impregnated mupirocin against S. aureus MRSA 15187 infected skin wound by using a dermal mouse model of 4-6-week-old BALB/c mice.

In yet another embodiment, the production of bacterial cellulose membranes is optimized in a cost-effective manner. The process is developed for efficient drug impregnation of Mupirocin (50 μg (0.00018%) to 500 μg (0.0162%)) on bacterial cellulose membranes and subsequent release study for the development of transdermal patches. Pharmacokinetic studies of mupirocin impregnated BCM was carried out and found that mupirocin was released from the patches to the application site followed by detection in plasma. Results of histopathological examination of the organs like liver, kidney, heart, and brain in acute dermal toxicity of mupirocin impregnated BCM showed no significant changes in the treatment group as compared to control group. The patch is designed in such a way that the medication permeates the skin in a controlled fashion thus attains more steady levels of the drug in the body.

In still another embodiment, Komagataeibacter hansenii (MBS-8) producing bacterial cellulose membrane for transdermal drug delivery was isolated and deposited at MTCC having accession number MTCC 13036.

In yet another embodiment, the isolated bacterial strain is a potent bacterial cellulose membrane producing strain for the drug impregnation and to develop transdermal patches.

In still another embodiment, the strain is isolated from rotten apple in saline water by incubating the beaker at a temperature of 25° C. for 15 days under static culture conditions.

In yet another embodiment, process engineering for the development of bacterial cellulose patches of desired thickness were developed. The said process comprising the following steps: the bacterial isolate of Komagataeibacter hansenii (MBS-8) was grown under static culture conditions in the production medium having two different carbon sources (glucose and glycerol) 2% to 5.0%; 0.5% to 1.0% of a nitrogen source (peptone and yeast extract); 0.1% to 0.25% of salts for 12 to 15 days; at pH 6.0-7.0; temperature ranges from 25 to 35° C.

In still another embodiment, effective downstream process was developed that leads to harvest bacterial cellulose membranes with the least shearing of the produced cellulose fibril. Biocellulose membranes were purified by NaOH and acetic acid prior to determination of dry weight followed by autoclaving for decontamination.

In yet another embodiment, impregnation of topical antibiotics Mupirocin (50 μg (0.00018%), 250 μg (0.0081%) & 500 μg (0.0162%)) per patch of the biocellulose membranes was done to develop dermal patches. Weight and drug content of mupirocin impregnated BCM were found to be uniform.

In still another embodiment, based on thermal and spectroscopic characterization, no possible interaction was observed due to impregnation of mupirocin in BCM. Permeation study of the drug Franz diffusion & impregnated BC membrane was done using agar diffusion assay cells.

In yet another embodiment, the in-vivo efficacy of the combination was tested in an infected mouse model of S. aureus infection. Treatment started at 24 hr. post-infection with antibiotic impregnated cellulose membrane. Quantitative estimation of bacterial load of the infected area was determined to calculate the number of CFU per infected skin patch.

In still another embodiment, in an in-vivo experiment the bacterial CFU load in the skin patch after 24 hrs. The establishing infection was 3.5×10⁷. Cellulose membranes impregnated with mupirocin (50 μg (0.00018%)/patch) showed 0.5 log CFU reduction on 5th day of treatment. Cellulose membranes were impregnated with mupirocin (250 μg (0.0081%)/patch) showed 1.5 log CFU reduction on 5th day of treatment. The drug on cellulose membrane patches was then increased to 500 μg (0.0162%)/patch and the Cellulose membranes was applied to the infected part after 24 hrs. to establish infection. In this experiment there was 2.5 log CFU reduction in treatment group on 5th day. The CFU count for untreated group was 8×10⁷ at the end of the day of experiment.

In yet another embodiment, pharmacokinetic studies of mupirocin-impregnated BCM was carried out in Balb/c mice using abraded skin model and it was found that mupirocin was released from the patches to the application site followed by detectable limits in plasma. Acute dermal toxicity of mupirocin-impregnated BCM was carried out in Wistar rats and it was found that there were no notable clinical signs of toxicity and mortality in the experimental animals as well as no marked changes were observed in relative organ weight, hematological parameters and biochemical parameters in treatment group as compared to control group.

In yet another embodiment results of histopathological examination of the organs like liver, kidney, heart, and brain in acute dermal toxicity of mupirocin-impregnated BCM showed no significant changes in the treatment group as compared to control group.

In an embodiment, the present invention provides a composition for antibacterial bio-cellulosic patches comprising:
[a] bacterial cellulose (BC) membrane;
[b] mupirocin in the range of 0.00018% to 0.033% taken from mupirocin stock solution of concentration 50 mg/ml dissolved in methanol; and
[c] glycerol in the range of 1.0% to 3.0%;
wherein [b] and [c] are loaded onto [a].

In an embodiment, the present invention provides a composition for antibacterial bio-cellulosic patches comprising:

$$\begin{matrix}bacterial cellulose membrane ; ( 3100000 μg = 3100 mg constant = 100 \% ) & [ a ] \\ mupirocin in the range of 50 μg = 0.05 mg = 0.00018 \% \\ 250 μg = 0.25 mg = 0.0081 \% \\ 500 μg = 0.5 mg = 0.0162 \% \\ 750 μg = 0.75 mg = 0.023 \% \\ 1000 μg = 1. mg = 0.033 \% & [ b ]\end{matrix}$$

taken from mupirocin stock solution of concentration 50 mg/ml dissolved in methanol
[c] glycerol in the range of 1.0%, 1.5%, 2.0%, 2.5%, 3.0% dissolved in 500 μl of buffer wherein [b] and [c] are loaded onto [a].

In another embodiment, the present invention provides a composition for antibacterial bio-cellulosic patches, wherein 500 μg mupirocin is loaded onto 3100 mg of the bacterial cellulose membrane.

In still another embodiment, the present invention provides a composition for antibacterial bio-cellulosic patches, wherein 0.0162% mupirocin is loaded onto the bacterial cellulose membrane.

In yet another embodiment, the present invention provides a composition for antibacterial bio-cellulosic patches, wherein 2.5% glycerol is loaded onto the bacterial cellulose membrane.

In another embodiment, the present invention provides a process for the preparation of the composition for antibacterial bio-cellulosic patches, wherein the steps comprising:
a) Culturing the isolated bacterial strain of Komagataeibacter hansenii (MBS-8) designated as MTCC 13036 in M5 medium comprising glucose 0.5%, glycerol 4%, peptone 0.5%, yeast extract 0.25%, disodium hydrogen phosphate 0.27%, citric acid 0.015% having pH in the range of 5.5 to 6.2 at a temperature ranging from 28 to 30° C. for a period of 7 to 9 days under static conditions to obtain bacterial cellulose membrane/bio-cellulosic patch on the surface of the medium;
b) The BC membrane obtained in step [a] was washed with boiled 1N NaOH having a temperature in the range of 80 to 90° C. for a period of 1 to 2 hr. and then washed with weak acid followed by washing with distilled water for 2-3 times until the pH becomes neutral;
c) The washed BC membrane obtained in step [b] was dipped in distilled water and autoclaved at a temperature of 115 to 120° C. for 15 to 20 minutes to obtain a sterilized BC membrane;
d) The sterilized bacterial cellulose membrane was weighed and then compressed by hands between two acrylic plates for the removal of 50-60% of their water content to obtain a drained BC membrane;
e) the drained BC membrane obtained in step [d] was soaked in potassium phosphate buffered solution having pH=7.4 containing mupirocin in the range of 50 to 1000 microgram and glycerol in the range of 0.5 to 5.0% for a duration of 24 to 48 hours at room temperature to assure complete absorption of the drug onto the membrane;
f) After the drug absorption, the antibacterial bio-cellulosic patches/membranes obtained were dried at temperature ranging from 30 to 40° C. in a ventilated oven for 10 to 16 hours to obtain the desired antibacterial bio-cellulosic patches/membranes.

In still another embodiment, the present invention provides a process, wherein culturing of the isolated bacterial strain MTCC 13036 is done at a temperature of 28° C. for 8 days.

In yet another embodiment, the present invention provides a process, wherein the weak acid is glacial acetic acid.

In a further embodiment, the present invention provides a composition for antibacterial bio-cellulosic patches comprising:
[a] Bacterial cellulose membrane;
[b] Mupirocin in the range of 0.00018% to 0.033% taken from mupirocin stock solution of concentration 50 mg/ml dissolved in methanol;
[c] Glycerol in the range of 1.0% to 3.0%;
   wherein [b] and [c] are loaded onto [a].

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 represents pharmacokinetic study for mupirocin impregnated BC membrane was carried out to obtain the mean plasma concentration of mupirocin in BALB/c MICE to detect mupirocin for assuring the release of the same on the application site.

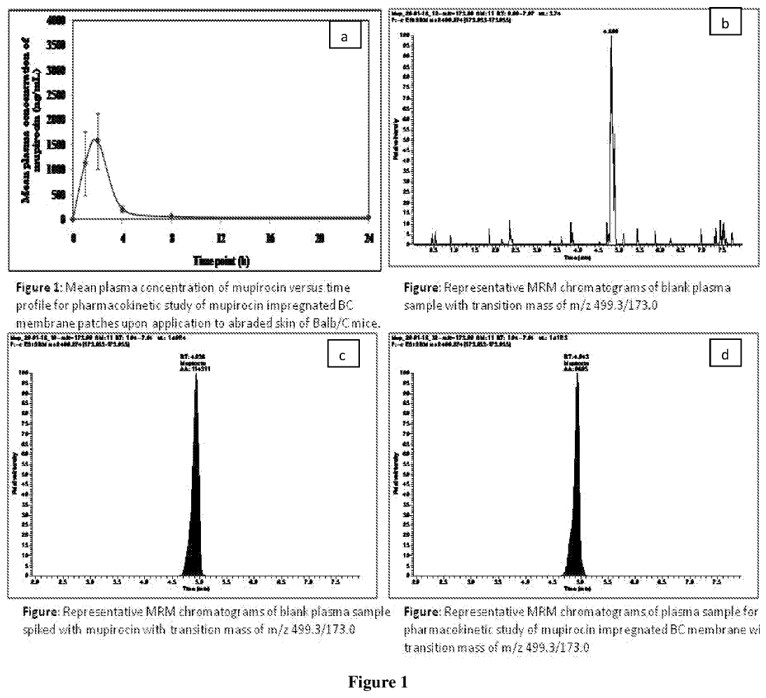

FIG. 2 represents the Body weight changes of the animals in the control group and treatment group for evaluating acute dermal toxicity of BC-impregnated with mupirocin.

FIG. 3 represents the in-vivo efficacy of cellulose membranes alone and in combination with impregnated mupirocin at different concentration along with % equivalent mupirocin ointment, 2% mupirocin ointment and placebo groups respectively were used in all the experiments.

A. Mupirocin 50 μg/patch impregnated in cellulose membrane.
B. Mupirocin 250 μg/patch impregnated in cellulose membrane
C. Mupirocin 500 μg/patch impregnated in cellulose membrane

### LIST OF ALL THE ABBREVIATIONS USED IN THE INVENTION

| | |
|---|---|
| BC | Bacterial cellulose |
| BCM | Bacterial cellulose membrane |
| MRSA | Methicillin resistance Staphylococcus aureus |
| Nm | Nanometer |
| μg | microgram |
| CFU | Colony forming units |
| MTCC | Microbial type culture collection |
| Log | Logarithm |
| FDA | Food and drug administration |
| SD | Standard deviation |
| Min | Minute |
| mg | Milligram |
| BCP | Bacterial cellulose patches |
| ml | Milliliter |
| Mup | Mupirocin |
| MHA | Mueller Hinton agar |
| EDTA | Ethylenediamine tetra acetic acid |
| OECD | Organization for Economic Cooperation and Development |
| DSC | Differential Scanning Calorimetry |
| FTIR | Fourier- Transform Infrared Spectroscopy |
| LC-MS | Liquid Chromatography - Mass Spectroscopy |
| ESI | Electron Spin Ionization |

The terms ‘mupirocin loaded bacterial cellulose membranes’ and ‘antibacterial bio-cellulosic patches’ as used in the present invention carry the same meaning. The terms ‘bacterial cellulose membrane’, ‘cellulose membrane’, ‘BC membranes’, ‘biocellulosic membrane’ and ‘biocellulose membrane’ have been used in the specification interchangeably. Likewise, the terms ‘composition’ and ‘formulation’ are used interchangeably.

### DETAILS OF BIOLOGICAL RESOURCES USED IN THE INVENTION

Samples for the isolation of bacterial cellulose producers were collected from waste dump area of fruits and vegetables market of Bakshi Nagar, Near Govt. Medical College, Jammu, 180001 Jammu and Kashmir, India.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of 35-50° C. should be interpreted to include not only the explicitly recited limits of 35° C.-50° C. but also to include sub-ranges, such as 41-49° C., and so forth, as well as individual amounts, within the specified ranges, such as 35.2° C., 40.5° C., and so on.

In the present invention, completely biodegradable antibacterial bio-cellulosic patches/antibacterial bacterial cellulose membranes were produced via fermentation with reference to the already available chemically synthesized patches in the market that are not environment friendly and are no/less biodegradable and may cause skin irritation and allergy. The instant invention used patches having biological origin and which are environment friendly. The produced bacterial cellulose has no toxicity and is generally regarded as safe by FDA.

The term ‘antibacterial bio-cellulosic patches’ as used in the present invention implies bacterial cellulose membranes loaded with Mupirocin dissolved in methanol, potassium phosphate buffer and has glycerol as plasticizer.

Screening for bacterial cellulose producers was carried out by isolation of bacterial isolates from the rotten fruits and vegetables. A total of 250 bacterial isolates were isolated. These isolates were numbered from MBS-1 to MBS-250 and were screened for their potential to produce bacterial cellulose. Results showed that out of the 250 bacterial isolates obtained, only 4 isolates produced bacterial cellulose. All these bacterial isolates were gram-negative rods. Among these 4 isolates MBS-1, MBS-8, MBS-54 and MBS-88 were considered for further studies.

In an embodiment of the present disclosure there is reported a composition for Bacterial Cellulose [BC] membranes in the form of patches having a definite amount of antibiotic mupirocin along with other ingredients which gets released form membrane when applied on wound infections.

TABLE 1
Illustrating the cellulose production capacity
of the 4 chosen isolated bacterial strains:

| | Temperature | | Bacterial | BC production |
|---|---|---|---|---|
| Medium | [° C.] | pH | strain | [g/l] |
| M2 | 25° c. | 5.8 | MBS-1 | 0.021 |
| M5 | 28° c. | 6 | MBS-8 | 2.843 |
| M4 | 30° c. | 6.2 | MBS-54 | 0.132 |
| M3 | 28° c. | 5.5 | MBS-88 | 2.12 |

As evident from the above Table, the best BC producer i.e., MBS-8 was chosen for all further studies and experiments. It was identified as Komagataeibacter hansenii based on 16 s-rRNA gene sequence and deposited with MTCC, IMTECH, Chandigarh India on Jun. 10, 2020 vide Accession No. MTCC-13036.

Impregnation of topical antibiotics in the bacterial cellulose membranes for the development of antibacterial bio-cellulosic dermal patches was carried out by the preparation of mupirocin loaded bacterial cellulose membranes along with other ingredients, essentially glycerol. The mupirocin solutions were prepared at 0.00018%, 0.0081% and 0.0162% (w/v) concentrations.

Loopful of bacterial colonies of the bacterial isolated strain of MBS-8 i.e., Komagataeibacter hansenii MTCC 13036 was transferred from agar plate into autoclaved broth having Glucose 0.5%, glycerol, 4% as carbon source, peptone 0.5%, yeast extract 0.25% as nitrogen source and disodium hydrogen phosphate 0.27%, citric acid-0.015% as salts having pH 6 and incubated under static conditions at a temperature of 28° C. for 4-5 days to obtain inoculum.

250 ml flasks having same medium were first autoclaved at 121.5° C. for 15 minutes and then inoculated with 1.5% of the developed inoculum concentration under laminar air flow and incubated at static conditions at 28° C. for 7-8 days incubation time. Then the BC membranes produced in the culture medium were taken out, washed with boiled 1N NaOH for 1 hour and then washed with weak acid namely glacial acetic acid to remove the medium components followed by washing with distilled water 2-3 times until the pH becomes neutral. Lastly, the cellulose membranes were dipped in distilled water and autoclaved at 121.5° C. for 15 minutes for further use. Freshly prepared wet BC membranes were immersed for 24 hrs. in the mupirocin solution having potassium phosphate neutral buffer (pH=7.4) as neutral buffer minimizes the effect of any H⁺ and OH⁻ ions on absorption of antibiotic in bio cellulose membranes. Although dry BC membranes have better stability and longer storage time, they are poor in permeability and absorption. To solve the problems with dry BC, plasticizers were used especially glycerol@2.5% was used as plasticizer due to its high plasticizing capacity and thermal stability at processing temperatures as it decreases the strength of intermolecular hydrogen bonds between adjacent cellulose chains that results in improvement of the flexibility of pellicles and prevention of formation of rigid bacterial cellulose membranes after drying. BC membranes loaded with mupirocin [BC/mupirocin] were dried at 40° C. for 24 hr. The sample of BC/mupirocin was taken into Eppendorf and methanol was added. AS mupirocin gets dissolved into methanol and shows minimum interaction with bacterial cellulose patches which was further shaken for 4 hr. for the maximum release of mupirocin. The sample was centrifuged and diluted with methanol to inject into the HPLC system to estimate mupirocin in each patch.

Contents of mupirocin in each impregnated BC membrane was evaluated to assess the actual individual content of mupirocin impregnated in the prepared antibacterial bio-cellulosic patches. Two batches of mupirocin impregnated BC membranes containing five numbers each were analyzed and by using standard calibration curve for mupirocin the content of mupirocin was calculated. Results showed Mupirocin content (mean±SD) for Batch-1 (μg/patch) was found to be 583±15. Mupirocin content (mean±SD) for Batch-2 (μg/patch) was found to be 590±19. Batch 1, 2 represent the experiment performed in duplicate and a mean of 5 membranes impregnated with same concentration were considered to find out the content of mupirocin/patch and units of mupirocin content are μg/patch

In an aspect, the present invention provides production and purification process of biocellulosic membranes which are developed in such a way that the produced membranes are of desired thickness and length, suitable for drug impregnation in a cost-effective manner for the development of transdermal patches.

In another aspect, the present invention provides a process for efficient drug impregnation of Mupirocin (50 μg (0.00018%) to 500 μg (0.0162%)) in bacterial bio-cellulosic patches and study the subsequent drug release for the development of antibacterial transdermal patches. Weight and drug content of mupirocin-impregnated BC patches were found to be uniform.

In yet another aspect, of the present invention based on thermal and spectroscopic characterization, there was no possible interaction was observed due to impregnation of mupirocin in BC Patches.

In still another aspect of the present invention, pharmacokinetic studies of mupirocin impregnated BCP was carried out in BALB/c mice using abraded skin model and it was found that mupirocin was released from the patches to the application site with detectable limits in plasma.

In yet another aspect of the present invention, acute dermal toxicity of mupirocin-impregnated BCM was carried out in Wistar rats, and it was found that there were no notable clinical signs of toxicity and mortality in the experimental animals as well as no marked changes in relative organ weight, hematological parameters and biochemical parameters were observed in treatment group as compared to control group.

In still another aspect of the present invention, the results of histopathological examination of the organs like liver, kidney, heart, and brain in acute dermal toxicity of mupirocin impregnated BCP and it was showed that there were no significant changes were observed in the treatment group as compared to control group.

In an embodiment of the present disclosure, there is provided a composition for antibacterial bio-cellulosic patches comprising:
[a] bacterial cellulose (BC) membrane;
[b] mupirocin in the range of 0.00018% to 0.033% taken from mupirocin stock solution of concentration 50 mg/ml dissolved in methanol; and
[c] glycerol in the range of 1.0% to 3.0%;
wherein [b] and [c] are loaded onto [a].

In an embodiment of the present disclosure, there is provided a composition for antibacterial bio-cellulosic patches as disclosed herein, wherein 500 μg mupirocin is loaded onto 3100 mg of the bacterial cellulose membrane.

In an embodiment of the present disclosure, there is provided a composition for antibacterial bio-cellulosic patches as disclosed herein, wherein 0.0162% mupirocin is loaded onto the bacterial cellulose membrane.

In an embodiment of the present disclosure, there is provided a composition for antibacterial bio-cellulosic patches as disclosed herein, wherein 2.5% glycerol is loaded onto the bacterial cellulose membrane.

In an embodiment of the present disclosure, there is provided a process for the preparation of the composition for antibacterial bio-cellulosic patches as disclosed herein, wherein the steps comprising:
(a) culturing the isolated bacterial strain of Komagataeibacter hansenii (MBS-8) designated as MTCC 13036 in M5 medium comprising glucose 0.5%, glycerol 4%, peptone 0.5%, yeast extract 0.25%, disodium hydrogen phosphate 0.27%, citric acid 0.015% having pH in the range of 5.5 to 6.2 at a temperature ranging from 28 to 30° C. for a period of 7 to 9 days under static conditions to obtain a bacterial cellulose membrane/bio-cellulosic (BC) patch on the surface of the medium;
(b) The BC membrane obtained in step [a] was washed with boiled 1N NaOH having a temperature in the range of 80 to 90° C. for a period of 1 to 2 hr. and then washed with a weak acid followed by washing with distilled water for 2-3 times until the pH becomes neutral;
(c) the washed BC membrane obtained in step [b] was dipped in distilled water and autoclaved at a temperature of 115 to 120 degree C. for 15 to 20 minutes to obtain a sterilized BC membrane;
(d) the sterilized bacterial cellulose membrane was weighed and then compressed by hands between two acrylic plates for the removal of 50-60% of their water content to obtain a drained BC membrane;
(e) the drained BC membrane obtained in step [d] was soaked in potassium phosphate buffered solution having pH 7.4 containing mupirocin in the range of 50 to 1000 microgram and glycerol in the range of 0.5 to 5.0% for a duration of 24 to 48 hours at room temperature to assure complete absorption of the drug onto the membrane;
(f) after the drug absorption, the antibacterial bio-cellulosic patches/membranes obtained were dried at temperature ranging from 30 to 40° C. in a ventilated oven for 10 to 16 hours to obtain the desired antibacterial bio-cellulosic patches/membranes.

In an embodiment of the present disclosure, there is provided a process as disclosed herein, wherein culturing of the isolated bacterial strain MTCC 13036 is done at a temperature of 28° C. for 8 days.

In an embodiment of the present disclosure, there is provided a process as disclosed herein, wherein the weak acid is glacial acetic acid.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The following examples are given by way of illustration only and therefore should not be construed to limit the scope of the present invention in any manner.

### Example 1

### Isolation, Screening, and Characterization of Bacterial Cellulose Producer.

Isolation of bacterial cellulose producer was carried from rotten fruits and vegetables and lab stock cultures. Isolates were kept in saline solution for 15 days at static state at 30° C. A white membrane sheet was observed over the top of flask containing apple residue and then culture was purified by streaking. The culture was then inoculated in Hestrin-Schramm medium (which is a reported cellulose producing medium) containing Glucose—20 gm; Peptone—5 gm; Yeast—5 gm; disodium hydrogen phosphate—2.7 gm; and Citric acid—1.15 gm; per 1 liter medium for cellulose production and calcium carbonate ethanol medium was used for calcium carbonate utilization which is the characteristic property of Acetobacter family. Then the culture was characterized by Electron microscopy and other biochemical standard tests to identify the same up to the species level and then it was deposited with the International Depository Authority MTCC, IMTECH, Chandigarh India under the Budapest Treaty.

### Example 2

### Production and Optimization of Bacterial Cellulose Producer Different Medium

Before optimizing the culture conditions, cellulose production was analyzed in static and agitation/shake flask conditions. Cellulose production was observed to be more in static conditions as compared to shaking mode. All the different medium in flasks in triplicates were autoclaved and inoculated with 1.5% of inoculum from single flask and incubated for 6 days at a temperature of 28±2° C. Then best medium giving highest cellulose yields was selected that is M5 having two carbon sources that is Glucose and glycerol and peptone and yeast extract as nitrogen source and disodium hydrogen phosphate, citric acid.

TABLE 2
Illustrating the Composition of Different Medium used for standardizing
the medium for best bacterial cellulose production [gm/liter]

| M1 | M2 | M3 | M4 |
|---|---|---|---|
| Glucose-56 | B-Glucose-20 | Glucose-18 | Sucrose- 50 |
| Yeast- 9.9 | Peptone-5 | Sucrose- 21 | Yeast-5 |
| Ethanol-17.2 | Yeast-5 | Corn steep liquor-20 | (NH₄)₂SO₄-5 |
| Beef extract -8 | Na₂HPO₄-2.7 | (NH₄)₂SO₄-4 | K₂HPO₄-3 |
| | Citric acid- 1.15 | K₂HPO₄-2 | MgSO₄- 0.15 |
| | | MgSO₄-0.4 | |
| M5 | M6 | M7 | M8 |
| Glucose-5 | Glucose-20 | Luria bertani-20 | I- Mannitol-15 |
| Peptone-5 | Peptone-5 | Glucose-3.6 | Tryptone-6 |
| Yeast Extract 2.5 | Yeast-5 | NaCl-0.5 | C₂H₅OH-7 |
| Glycerol-40 | Na₂HPO₄-2.7 | KCl-0.18 | Acetic acid -2 |
| Na₂HPO₄-2.7 | Citric acid- 1.15 | MgCl₂-0.95 | Nicotinic acid-0.002 |
| Citric acid-0.15 | C₂H₅OH-40 | MgSO₄-2.5 | CaCl₂-0.1 |
| M9 | M10 | M11 | M12 |
| Glucose-10 | Glucose-15 | Glucose-15 | Fructose-30 |
| Peptone-7 | Peptone-3 | Corn steep liquor-20 | Corn steep liquor-10 |
| Yeast-10 | Yeast-3 | (NH₄)₂SO₄ -2 | K₂HPO -2 |
| Acetic acid-1.5 | C₂H₅OH-5 | K₂HPO₄-3 | MgSO₄ -0.25 mg |
| Succinic acid-0.16 | NaCl-0.1 | Na₂HPO₄-3 | (NH₄)₂SO₄ -3.3 mg |
| C₂H₅OH -10 | Acetic acid -3 | MgSO₄-0.80 | FeSO₄ -3.6 mg |
| | Folic acid-0.0004 | FeSO₄-0.005 | CaCl₂ -14.7 mg |
| | | H₃BO₃-0.003 | Na₂MoO₄ -2.42 mg |
| | | C₆H₆N₂O-0.05 | ZnSO₄ -1.73 mg |
| | | | MnSO₄ -1.39 mg |
| | | | CuSO₄ -0.05 mg |
| | | | Inositol -0.2 mg |
| | | | Nicotinic acid -0.04 mg |
| | | | Pyridoxine -0.04 mg |
| | | | Thiamine-0.04 |
| | | | Calcium pantheonate-0.02 mg |
| | | | Riboflavin -0.02 mg |
| | | | Para amino benzoic acid -0.02 mg |
| | | | Folic acid -0.0002 mg |
| | | | Biotin - 0.0002 mg |

TABLE 2
Depicting Yield of Bacterial cellulose
obtained in 1 liter of different medium

| Medium | Yield of BC [g/l] |
|---|---|
| M1 | 4.513 |
| M2 | 4.965 |
| M3 | 1.298 |
| M4 | 2.904 |
| M5 | 5.967 g/l |
| M6 | 3.84 |
| M7 | 2.101 |
| M8 | 2.8908 |
| M9 | 2.03 |
| M10 | 2.7 |
| M11 | 1.254 |
| M12 | 2.358 |

The above Tables represents the analysis of 12 different medium (M1 to M12) reported in the literature for maximum bacterial cellulose production. Results shows that medium M5 supported maximum production of 5.967 g/l when Glucose (0.5%-2%) and glycerol (2%-4%) were used as carbon source and Yeast (0.25%-1%) and peptone (0.5%-1%) was used as nitrogen source.

TABLE 4
Physical and nutritional parameters optimization
for production of bacterial cellulose and scaling
of cellulose production up to 1 liter.

| Factors | Optimized condition | |
|---|---|---|
| Medium | Glycerol (M5) | |
| Agitation rate | Static cultivation | |
| pH | 5.5-6.2 | |
| Temperature | 28° C.-30° C. | |
| Incubation period | 7-9 | days |
| Inoculum size | 1.5%-2% | |
| Carbon source and its concentration | Glucose- 0.5%-2%, | |
| | glycerol2%-4% | |
| Nitrogen source and its concentration | Peptone- 0.5%-1% | |
| | yeast extract-0.25-1% | |
| Initial Yield (unoptimized condition) | 0.528 | g/l |
| Yield in standard control medium | 5.967 | g/l |
| Final Yield (after standardization | 14.235 | g/l |
| of all the variables) | | |

The above Table represents the final optimization conditions obtained after one variable at a time optimization procedure. Herein 14.235 g/l of bacterial cellulose was produced when Medium (M5) containing glucose and Glycerol, 5.967 g/l in Standard (Hestrin schramn) medium and 14.235 g/l after optimizing the physical and nutritional parameters.

Different parameters were optimized starting from temperature, pH, Incubation time, age of inoculum and inoculum size. Results shows static environment, temperature of 28° C.-30° C., pH of 5.5-6.2, inoculum concentration of 1.5%-2% incubation time of 7-9/10 days and inoculum age of 4-6, 5 days were found to be best for maximum cellulose production in M5 medium. The medium (M5) resulting in maximum production of cellulose in 100 ml flasks was used in all further experiments for production of bacterial cellulose.

### Example 3

### Preparation of Antibacterial Cellulose Membranes

A loopful of bacterial colonies grown on agar plate of M5 medium were transferred into autoclaved M5 broth having glucose 0.5%-2% glycerol 2%-4% as carbon source, peptone 0.5%-1% yeast extract 0.25%-1% as nitrogen source and disodium hydrogen phosphate 0.27%-0.50% citric acid—0.015%-0.025% as salt having pH 5.5-6.2.

Inoculated medium was incubated under static conditions at a temperature of 28° C.-30° C. to achieve 4-6 days inoculum age. Then 250 ml flasks having M5 medium were inoculated with 1.5%-2% inoculum concentration under laminar air flow. Flasks were incubated under static conditions at a temperature of 28° C.-30° C. for 7-9 days incubation time; after which a mat was seen on the surface of the medium. This mat was the cellulose membrane/bio-cellulosic patch.

The synthesized BC membranes were taken out, washed with boiled 0.5N-1N NaOH having a temperature of 80° C.-90° C. for 1-2 hr. and then washed with weak acid preferably glacial acetic acid to remove the medium components followed by washing with distilled water for 2-3 times until the pH becomes neutral.

For in-vivo and in vitro studies, the BC membranes were dipped in distilled water and were autoclaved at a temperature of 121.5° C. for 15 minutes for further use.

### Example 4—Process, Selection and Optimization of Different Ranges of the Selected Components for Impregnation in Antibacterial Bio-Cellulosic Patches

### Composition of the Developed Antibacterial Bio-Cellulosic Patches:

a) Bacterial cellulose membranes
b) Mupirocin
c) Glycerol

### Process of Impregnation of the Antibiotic in BC Patches

1. Wet circular Bacterial cellulose membranes were weighed.
2. These membranes were then compressed by hands between two acrylic plates for the removal of 50-60% of their water content.
3. Drained BC membranes were soaked in a potassium phosphate buffered solution (pH 7.4) containing mupirocin (50 to 1000 microgram) and glycerol (0.5% to 4% v/v of glycerol to the buffer) for a duration of 40 to 48 hours at room temperature to assure complete absorption of the drug.
4. After the drug absorption, the antibacterial bio-cellulosic patches/membranes obtained were dried at temperature of 30° C. to 40° C. in a ventilated oven for 10 to 16 hours.
5. Dried antibacterial bio-cellulosic patches/membranes were kept in a desiccator until further use.

TABLE 5
Ranges of the components impregnated in
the antibacterial bio-cellulosic patches

| | | | Best Selected |
|---|---|---|---|
| Sl. | | | Range after |
| No. | Component | Different Ranges | in-vivo studies |
| 1 | BC patch | Plain BC patches | No activity |
| 2 | Mupirocin | 50 ug, 250 ug, 500 ug, | 500 ug |
| | | 750 ug, 1000 ug | |
| 3 | Glycerol | 0.5%, 1.0%, 1.5%, 2.0%, 2.5%, | 2.5% |
| | | 3.0%, 3.5%, 4.0%, 4.5%, 5.0% | |

### Release Study of Different Concentration of Mupirocin at Different Time Intervals

Table 6 represents the BC containing different concentration of mupirocin and the release of the same during the course of time

| Time | Mup | Mup | Mup | Mup | Mup |
|---|---|---|---|---|---|
| (hrs.) | 50 ug | 250 ug | 500 ug | 750 ug | 1000 ug |
| 0 h | 0 | 0 | 0 | 0 | 0 |
| 5 h | 45.34 | 63.73 | 74 | 65 | 84 |
| 10 h | 45 | 106.3 | 168 | 189 | 197 |
| 20 h | 46 | 169.4 | 230 | 276 | 325 |
| 30 h | 46 | 231 | 375 | 380 | 481 |
| 40 h | 46 | 246 | 480 | 476 | 563 |
| 50 h | 46 | 246 | 489 | 580 | 680 |

Table represents that time taken by patch containing 50 μg (0.00018%), 250 μg (0.0081%), 500 μg (0.0162%), 750 μg (0.0230%) and 1000 μg (0.033%) of mupirocin for maximum release were found to be 20 hr., 40 hr., 50 hr., 50 hr. and 50 hr. respectively.

TABLE 7
Different concentration of glycerol used as a plasticizer
along with BC patch having mupirocin

| Glycerol concentration | Mupirocin patches |
|---|---|
| (%) | (500 μg) |
| 0 | 110 |
| 0.5 | 176 |
| 1.0 | 230 |
| 1.5 | 276 |
| 2.0 | 463 |
| 2.5 | 497 |
| 3.0 | 488 |
| 3.5 | 462 |
| 4.0 | 473 |
| 4.5 | 488 |
| 5.0 | 489 |

The above Table represents that 2.5% of glycerol was adequate to provide the desired elasticity and in turn the release of maximum mupirocin.

TABLE 8
Composition of Antibacterial Bio-cellulosic patches prepared with
different concentration of the ingredients along with their antibacterial
activity (*100% is equivalent to 2.5 log reduction)

| | | | | | Results/% log |
|---|---|---|---|---|---|
| | | Component A | | | reduction |
| | | (Bacterial | Component B | Component C | in growth |
| Sl. | | cellulose | (Mupirocin) | (Glycerol) | inhibition |
| No. | Composition | membrane) | w/v | v/v | of S. aureus |
| 1. | (A) | BC | — | | (0%) |
| | | Membrane | | | |
| 2. | (A + B + | BC | 0.00018% | 1.5% | (50%) |
| | C + D) | Membrane | | | |
| 3. | (A + B + | BC | 0.0081% | 2.0% | (70%) |
| | C + D + E) | Membrane | | | |
| 4. | (A + B + | BC | 0.0162% | 2.5% | (100%) |
| | C + D + E) | Membrane | | | |
| 5. | (A + B + | BC | 0.0230% | 3.0% | (90%) |
| | C + D + E) | Membrane | | | |
| 6. | (A + B + | BC | 0.0330% | 3.5% | (85%) |
| | C + D + E) | Membrane | | | |

The result of the above Table indicate that most efficient patches were made from the serial no. 4 wherein 2.5 log reduction (100%) of growth was observed when Component A (Bacterial cellulose patch), Component B (Mupirocin)—500 μg (0.0162%), and Component C (Glycerol)—2.5% were used to develop the antibacterial biocellulosic patch. It may be observed that when only Bacterial cellulose membrane was used no antibacterial activity was obtained.

### Characterization of Mupirocin-Impregnated Patches

### Example 5—Weight Variation Test

Weight variation of mupirocin-impregnated BC membranes was evaluated by individually weighing five numbers of equal size patches using electronic balance (Model: XS205DU; Make: Mettler Toledo). Then, uniformity in patch weight was assessed.

### Example 6—Drug Content Assay

Mupirocin-impregnated BC membrane was used to estimate the content of mupirocin in it. Mupirocin-impregnated BC membrane was taken individually into Eppendorf tube and then, 2 mL of methanol was added to it and kept under shaking for 50 h. The sample was then taken out followed by centrifugation at 5000 rpm for 10 min and diluted with methanol to inject into the HPLC system (Model: Ultimate 3000; Make: Thermo Fisher Scientific). Study was performed using six replicates and samples were analyzed by an earlier reported HPLC method for mupirocin with minor modifications [Amrutiya et al., 2010]. A stock solution of mupirocin was prepared in methanol and further diluted with methanol to prepare calibration standards for the determination of the content of mupirocin in each patch [Amrutiya et al., 2009].

### Example 7—Differential Scanning Calorimetry (DSC)

DSC analysis of mupirocin and mupirocin-impregnated BC membrane drug were carried out by using DSC instrument (Model: DSC 6000; Make: PerkinElmer). Sample (1-2 mg) was weighed directly in an aluminum pan, crimped firmly with the lid to provide an adequate seal and kept in the sample holder of the instrument for scanning in the temperature range of 30-250° C. The rate of heating was 10° C./min under nitrogen atmosphere where the flow rate of nitrogen gas was 20 mL/min. Data was evaluated using Pyris software [Ali et al., 2018; Nandi et al., 2018].

### Example 8—Thermal Gravimetric Analysis (TGA)

TGA analysis of mupirocin and mupirocin-impregnated BC membrane were carried out by using TGA instrument (Model: TGA/DSC1; Make: Mettler Toledo). Sample (1-5 mg) was weighed, kept in alumina crucible, and placed in sample holder for scanning in the range of 40-600° C. The rate of heating was 10° C./min under nitrogen atmosphere where the flow rate of nitrogen gas was 50 mL/min. Data was evaluated by Stare software [Ali et al., 2018; Nandi et al., 2018].

### Example 9—Fourier-Transform Infrared Spectroscopy (FTIR)

FTIR spectra of mupirocin and mupirocin-impregnated BC membrane were generated using FTIR instrument (Model: IRAffinity-1S; Make: Shimadzu). The spectrophotometer was equipped with an ATR cell and the spectrum was collected in the range of 700-2000 cm⁻¹ where spectral resolution of 4 cm⁻¹ were used to obtain good quality spectra. Data was analyzed by LabSolutions software [Ali et al., 2018; Nandi et al., 2018].

### Example 10-Scanning Electron Microscopy (SEM)

SEM image of mupirocin and mupirocin-impregnated BC membrane were obtained by using SEM instrument (Model: JSM-IT300LV; Make: Jeol). It was operating at 5 kV and magnification at 50×/500× as required [Durrigl et al., 2011].

### Example 11. Results of Characterization of Mupirocin-Impregnated Patch

Weight variation of five equal size patches (mean±SEM) were found to be 10.5±0.9 mg. Results revealed that mupirocin-impregnated BC membrane-maintained uniformity in weight. Content of mupirocin in each mupirocin-impregnated BC membrane was estimated using a HPLC method for estimating mupirocin. Mupirocin free acid content (mean±standard error mean) was found to be 590±34 μg in each patch.

DSC study was performed for both mupirocin and mupirocin-impregnated BC membrane to observe the thermal behavior of mupirocin in such form as compared to in patch. The DSC thermogram of mupirocin displayed a sharp endothermic peak corresponding to its melting point at 77.49° C. where onset temperature and end temperature was 74.24° C. and 80.95° C., respectively. The melting temperature is in line with the reported literature [Greenway et al., 1997; Amrutiya et al., 2009]. The heat of fusion was found to be 96 Jg⁻¹. There was the presence of another broad endothermic event of mupirocin as such form in the range of 147-158° C. (153.56° C.). In case of mupirocin in mupirocin-impregnated BC membrane, this event remained unaffected (150.47° C.) but sharp endothermic peak corresponding to its melting point presented a broad endothermic peak with the loss of its sharp appearance. Similar evidence was also reported for mupirocin [Amrutiya et al., 2009]. TGA analysis was performed for both mupirocin and mupirocin-impregnated BC membrane to observe the thermal behavior of mupirocin in as such form as compared to patch. There was around 94% mass loss was observed for mupirocin as such form in the temperature range of 90-526° C. Similar thermo analytical profile with around 93% mass loss was observed in case of mupirocin in mupirocin-impregnated BC membrane at broader temperature range (52-597° C.) as compared to mupirocin in its native form. There was also no marked mass loss observed due to thermal decomposition i.e., accelerated degradation which was observed during incompatibility.

FT-IR analysis was performed for both mupirocin and mupirocin-impregnated BC membrane to observe any interaction of mupirocin in as such form as compared to impregnated form in patch. The following peak positions for mupirocin were selected to evaluate the compatibility of mupirocin: 1712, 1716 and 1726 cm⁻¹ corresponds to C═O group (Range of wave number: 1700-1725 cm⁻¹; Type of vibration: strong stretching; Functional group: acid); 1222 and 1232 cm⁻¹ corresponds to C—O group (Range of wave number: 1163-1210 cm⁻¹; Type of vibration: strong stretching; Functional group: ester); 1143, 1161 and 1169 corresponds to C—O group (Range of wave number: 1050-1150 cm⁻¹; Type of vibration: strong stretching; Functional group: alcohol). There was no substantial difference in the above-mentioned peak positions of mupirocin while impregnated in patch. Therefore, FT-IR data eliminates any possible incompatibility of mupirocin in patch.

### Example 12—Permeation Study of the Drug Franz Diffusion &Impregnated BC Membrane Using Agar Diffusion Assay Cells

Methodology: In-vitro drug release study was carried out using mupirocin impregnated BC membrane disk. BCM discs was placed at the center of Mueller Hinton agar plate spreaded with 100 μl of S. aureus MRSA 15187 and was then incubated at 37° C. for 24 hours. Results showed a clearing of zone around the disc where antibiotic stopped the bacteria from growing or killed the bacteria. Release of mupirocin from the BC disc were able to inhibit the growth of bacteria around the disc and this zone can be increased on increasing the concentration of mupirocin.

TABLE 9

| | Zone of |
|---|---|
| Mupirocin (5 μg)/disc | clearance (mm) |
| Methicillin resistant staphylococcus aureus | 2.5 |
| 15187 | |
| Streptococcus pyogenes | 3 |
| Staphylococcus aureus ATCC 29213 | 2.8 |
| Control BC disc having no mupirocin lacks | nil |
| any zone of inhibition against pathogens | |

### Example 13

### Pharmacokinetic Study in Animal Model

Methodology: Pharmacokinetic study of mupirocin was carried out using mupirocin impregnated BC membrane patches in male Balb/c mice. Animals were divided into groups for sparse sampling technique. Animals were shaved on the dorsal side of the body to apply patches one day before experimentation. On the day of experimentation, shaved area in the animal was gently abraded with sandpaper and mupirocin impregnated BC membrane was applied with the help of backing membrane. Then, blood samples were collected in tubes containing aqueous EDTA solution (5%, w/v) at 0 hr., 1 hr., 2 hrs., 4 hrs., 8 hrs., and 24 hrs. Then, plasma was separated after centrifugation of blood samples at 3000 rpm and stored at −80° C. until analysis.

Samples were thawed on the day of sample analysis and then by plasma protein precipitation technique using methanol. A matrix match calibration curve of mupirocin was prepared by spiking the known amount of mupirocin into blank plasma followed by quantitation by LC-MS/MS. Separation was achieved in Chromolith high resolution RP18e column (50×4.6 mm) using isocratic mobile phase composition of acetonitrile and 0.1% (v/v) formic acid in water (80:20 v/v) with a flow rate of 0.2 mL/min. Quantitation of the compound was performed in negative mode using MS/MS equipped with ESI source. Detection of ion was performed in SRM mode with parent ion/product ion transitions of 499.3/173.0. Data obtained from sample analysis was further evaluated for mean plasma concentration of mupirocin at respective time points.

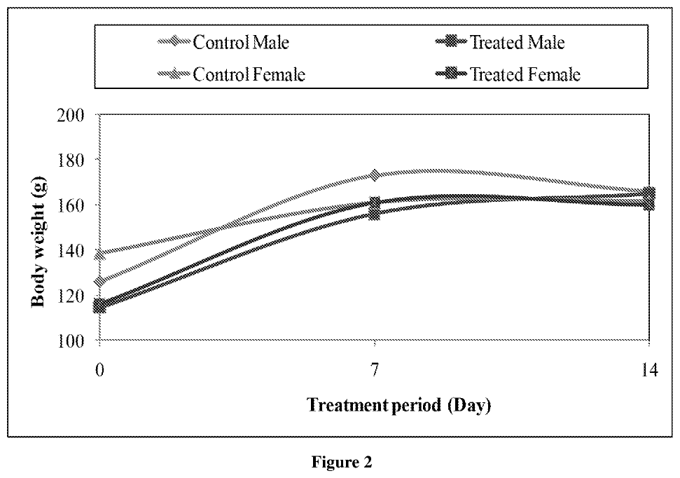

Results: Pharmacokinetic study for mupirocin impregnated BC membrane was carried out to obtain the mean plasma concentration of mupirocin at different time points (FIG. 2). Firstly blank plasma concentration was detected to check the presence of mupirocin due to any environmental factors then chromatograms of blank plasma spiked with known mupirocin was detected, also Chromatograms of mupirocin impregnated BC membrane was detected and at last Mean plasma concentration of mupirocin versus time profile of mupirocin impregnated BC membrane patches upon application to abraded skin of Balb/c mice was detected it was found that maximum concentration of mupirocin (ng/ml) in plasma detected 1576±567 upto 2 hours and then the concentration start decreasing from 1576±567 at 2 hours upto 38±16 at 24 hours.

TABLE 10

| | | Mean plasma concentration of |
|---|---|---|
| Serial | Time | mupirocin(ng/ml) (mean ± |
| no. | point(hr.) | Standard error mean) |
| 1 | 0 | 0 ± 0 |
| 2 | 1 | 1127 ± 645 |
| 3 | 2 | 1576 ± 567 |
| 4 | 4 | 210 ± 67 |
| 5 | 8 | 55 ± 29 |
| 6 | 24 | 38 ± 16 |

### Example 14—Bio-Evaluation of the Drug Impregnated BC Membrane; an Alternative Delivery System for the Particular Drug for Treatment of Infections Caused by the Particular Drug Susceptible Micro-Organisms

Methodology: Acute dermal toxicity of mupirocin impregnated BC membrane was carried out using Wistar rats adhering OECD guidelines after obtaining necessary approval from Institutional Animal Ethics Committee of CSIR-IIIM, Canal Road, Jammu, J&K, India. Animals were housed in polypropylene cages, kept at standard laboratory conditions (25±2° C., 50±20% relative humidity, 12 hr. light/12 hr. dark cycle), fed with standard pellet diet with water ad libitum. Animals were shaved on the dorsal side of the body to apply patches one day before the experimentation. On the day of experimentation, animals were divided into two groups namely control group and treated group containing six numbers of each male and female animals. BC membrane with or without impregnated mupirocin was applied on individual animals of control group and treated group, respectively. Then, animals were observed for any clinical signs of irritation, general behavior, toxicity, and mortality as well as food and water consumption daily for 14 days. Body weight of animals was measured on the day of experimentation and then, on weekly basis. After 14 days, blood samples were collected in tubes with EDTA as well as without EDTA from overnight fasted animals for evaluation of hematological and biochemical parameters, respectively. The following hematological parameters were evaluated in Table 12 viz. total white blood cell (WBC) count, WBC differential counts like lymphocyte, monocyte, neutrophil, eosinophil, and basophil counts, red blood cell (RBC) count, hemoglobin (Hb), hematocrit, mean corpuscular volume (MCV), mean corpuscular hemoglobin (MCH), platelet using automatic hematology analyzer (Sysmex XT 1800i, Transasia). The following biochemical parameters were evaluated in Table 13 viz. alkaline phosphatase (ALP), glucose, triglycerides (TG), cholesterol (CH), uric acid (UA), total protein (TP), alanine aminotransferase (ALT) and aspartate aminotransferase (AST) using automated clinical chemistry analyser (EM360, Erba Mannheim). Then, animals were sacrificed by cervical dislocation under anesthesia and followed by isolation of organs like brain, liver, kidney, heart which were rinsed, blotted dry, weighed, preserved in 10% formalin solution for histopathological examination. Statistical signification for treated group data was evaluated at p<0.05 level in comparison to control group data.

Results: Acute dermal toxicity of mupirocin impregnated BC membrane was investigated in comparison to blank BC membrane to identify any toxic effect. There were no notable clinical signs of toxicity and mortality in the experimental animals. Overall body weight of animals increased with respect of time in both the groups (FIG. 2). Though no marked changes were observed in relative organ weight (Table 11) there were some changes in hematological parameters (Table 12) and biochemical parameters (Table 13).

TABLE 11
Relative organ weight (organ to body weight ratio) in control
group and treated group of animals with both sexes for acute
dermal toxicity of mupirocin impregnated BC membrane

| Group | Sex | Brain | Heart | Lungs | Liver | Kidney |
|---|---|---|---|---|---|---|
| Control | Male | 0.92 ± 0.03 | 0.34 ± 0.01 | 0.56 ± 0.08 | 3.25 ± 0.11 | 0.34 ± 0.02 |
| | Female | 0.99 ± 0.02 | 0.34 ± 0.01 | 0.56 ± 0.02 | 3.39 ± 0.06 | 0.35 ± 0.01 |
| Treated | Male | 0.97 ± 0.02 | 0.32 ± 0.01 | 0.57 ± 0.06 | 3.20 ± 0.09 | 0.34 ± 0.01 |
| | Female | 1.00 ± 0.04 | 0.32 ± 0.00 | 0.59 ± 0.04 | 3.49 ± 0.12 | 0.36 ± 0.01 |

Footnote: Data are represented as Mean ± SEM.

TABLE 12
Hematological profile in control group and treated group of animals with
both sexes for acute dermal toxicity of mupirocin impregnated BC membrane

| | Control | | Treated | |
|---|---|---|---|---|
| Parameters | Male | Female | Male | Female |
| WBC (10³/μL) | 13.13 ± 0.28 | 9.78 ± 0.75 | 12.08 ± 0.46 | 9.47 ± 0.72 |
| RBC (10⁶/μL) | 6.91 ± 0.26 | 6.36 ± 0.25 | 6.95 ± 0.16 | 6.66 ± 0.16 |
| Hb (g/dL) | 13.67 ± 0.33 | 13.13 ± 0.56 | 13.25 ± 0.30 | 12.85 ± 0.31 |
| HCT (%) | 38.65 ± 0.74 | 38.27 ± 1.47 | 37.18 ± 0.76 | 36.87 ± 0.63 |
| MCV (fL) | 56.10 ± 1.27 | 60.35 ± 2.18 | 53.53 ± 0.42 | 55.48 ± 0.97 |
| MCH (pg) | 35.33 ± 0.34 | 34.30 ± 0.47 | 35.62 ± 0.35 | 32.20 ± 2.69 |
| Platelet (10³/μL) | 624.33 ± 70.0 | 639.00 ± 70.60 | 731.33 ± 35.31 | 455.00 ± 101.33 |
| Neutrophil (10³/μL) | 2.01 ± 0.19 | 0.86 ± 0.10 | 2.79 ± 0.24* | 1.25 ± 0.10^{#} |
| Lymphocytes (10³/μL) | 9.75 ± 0.28 | 7.64 ± 0.64 | 8.15 ± 0.43* | 7.20 ± 0.60 |
| Monocytes (10³/μL) | 1.02 ± 0.13 | 0.83 ± 0.13 | 0.85 ± 0.13 | 0.63 ± 0.11 |
| Eosinophils (10³/μL) | 0.32 ± 0.06 | 0.36 ± 0.05 | 0.28 ± 0.04 | 0.38 ± 0.04 |
| Basophils (10³/μL) | 0.02 ± 0.00 | 0.02 ± 0.01 | 0.01 ± 0.00 | 0.01 ± 0.00 |

Footnote:
Data are represented as Mean ± SEM; Statistical significance at p < 0.05 where *represents control male v/s treated male and ^{#}control female vs treated female.

TABLE 13
Biochemical profile in control group and treated group of animals with both
sexes for acute dermal toxicity of mupirocin impregnated BC membrane

| | Control | | Treated | |
|---|---|---|---|---|
| Parameters | Male | Female | Male | Female |
| ALP (U/L) | 340.83 ± 38.50 | 265.17 ± 22.06 | 340.50 ± 24.33 | 307.33 ± 14.44 |
| Glucose (mg/dl) | 95.23 ± 5.93 | 102.07 ± 3.05 | 97.02 ± 2.37 | 124.68 ± 4.38 |
| TG (mg/dl) | 87.27 ± 6.67 | 83.38 ± 6.44 | 91.28 ± 5.63 | 81.53 ± 4.36 |
| CH (mg/dl) | 72.33 ± 3.32 | 81.33 ± 4.32 | 75.33 ± 2.81 | 67.00 ± 2.67^{#} |
| UA (mg/dl) | 0.58 ± 0.05 | 0.90 ± 0.10 | 0.68 ± 0.05 | 0.85 ± 0.06 |
| TP (g/dl) | 6.72 ± 0.13 | 6.99 ± 0.11 | 6.27 ± 0.06* | 6.86 ± 0.17 |
| AST (U/L) | 146.57 ± 10.29 | 139.70 ± 6.08 | 145.53 ± 6.29 | 124.38 ± 11.04 |
| ALT (U/L) | 67.83 ± 6.42 | 57.70 ± 4.29 | 68.27 ± 3.96 | 63.82 ± 6.05 |

Footnote:
Data are represented as Mean ± SEM; Statistical significance at p < 0.05 where *represents control male versus treated male and ^{#}control female versus treated female.

### Example 15

### In-Vivo Efficacy of the Combination

The in-vivo efficacy of the combination was tested in a mouse model of infection. This study was approved by the Institutional Animal Ethics Committee (IAEC study no. SSP-0415, August 2009). In the study dermal infection model was developed on 2-3-weeks-old Balb/c mice weighing 20-22 g using MRSA 15187 strain of S. aureus as the infectious organism. The complete back side of the anaesthetized mouse was cleaned by removing hair and a blood oozing patch of 1 cm² was created by abrasion with sandpaper. A sterile cotton swab dipped in inoculum adjusted to a 0.5 McFarland standard (prepared from an overnight culture) was applied to the abraded area and left for 24 hr. to establish infection.

Treatment started at 24 hr. post-infection and three groups of mice were treated with impregnated cellulose membrane with Mupirocin at concentration of (50 μg (0.00018%), 250 μg (0.0081%) & 500 μg (0.0162%)/patch of cellulose membrane) and another group of infected mice were treated with cellulose membrane. Three experiments were performed by taking different concentrations of impregnated mupirocinas a treatment group. A group of infected mice without treatment were used as the untreated control and another group of mice were applied Mupirocin 2% and referred as positive control. Three mice were sacrificed humanely on daily basis for 5 consecutive days by CO₂ asphyxiation. The infected patch was aseptically dissected and homogenized in 1 ml normal sterile saline. Ten-fold Serial dilutions of the homogenates were plated in triplicate manner onto MHA plates supplemented with 2 μg/ml ciprofloxacin. These plates were incubated overnight at 37° C. and bacterial colonies were enumerated manually to calculate the number of CFU. per infected skin patch.

TABLE 14
Log 10 CFU values of in vivo experiment in consecutive 5 days.

| | | | | Mupirocin 500 μg/Patch of | | |
|---|---|---|---|---|---|---|
| Days | Mupirocin 0.5% | | | cellulose membrane | | |
| 0 | 7.875 | 7.3979 | 7.3979 | 7.875 | 7.3979 | 7.3979 |
| 1 | 6.845 | 6.4771 | 6.778 | 6.4771 | 6.6532 | 6.544 |
| 2 | 6.477 | 6.845 | 6.477 | 6.4771 | 6.301 | 6.301 |
| 3 | 6.301 | 6.176 | 6.301 | 5.845 | 5.954 | 5.176 |
| 4 | 6.177 | 6.176 | 6.544 | 5.845 | 5.301 | 5.397 |
| 5 | 6.477 | 5.954 | 6.875 | 4.845 | 5.301 | 4.698 |

Log 10 CFU values of in-vivo experiment in consecutive 5 days were recorded. In 0.5% Mupirocin group 3×10⁶ CFU was observed at the final day of experiment and in case of Mupirocin 500 μg/Patch of BC group 8×10⁴ CFU was found on the 5^{th} day.

TABLE 15

| | | | | Mupirocin 250 μg/Patch of | | |
|---|---|---|---|---|---|---|
| Days | Mupirocin 0.25% | | | cellulose membrane | | |
| 0 | 7.875 | 7.3979 | 7.3979 | 7.875 | 7.3979 | 7.3979 |
| 1 | 6.845 | 6.4771 | 6.778 | 6.845 | 7.177 | 6.778 |
| 2 | 6.477 | 6.845 | 6.477 | 6.4771 | 6.6532 | 6.544 |
| 3 | 6.845 | 6.845 | 7 | 6.4771 | 6.301 | 6.301 |
| 4 | 7 | 6.653 | 7.176 | 5.901 | 6.176 | 6.176 |
| 5 | 6.301 | 6.845 | 7.176 | 5.875 | 5.7781 | 6.176 |

Mup 500 μg (0.0162%)/Patch of BC group shows 2.5 log CFU reduction on the final day of experiment. Mupirocin 0.25% and Mup 250 μg (0.0081%)/Patch reduces the CFU load in a mice dermal model up to 0.5 to 1.5 log 10 CFU

TABLE 16

| | | | | Mupirocin 50 μg/Patch of | | |
|---|---|---|---|---|---|---|
| Days | Mupirocin 0.05% ointment | | | cellulose membrane | | |
| 0 | 7.875 | 7.3979 | 7.3979 | 7.875 | 7.3979 | 7.3979 |
| 1 | 7.698 | 7.176 | 7.3979 | 7.845 | 7.397 | 6.778 |
| 2 | 7.397 | 7.176 | 6.812 | 7 | 7.397 | 7.176 |
| 3 | 6.901 | 7 | 7.301 | 7.477 | 6.301 | 6.477 |
| 4 | 6.901 | 7 | 7.176 | 7.477 | 6.301 | 6.477 |
| 5 | 6.698 | 7.176 | 7.3979 | 7 | 6.176 | 6.176 |

Mupirocin 0.05% and Mup 50 μg (0.00018%)/Patch reduces the CFU load in a mice dermal model up to 0.5 log CFU.

TABLE 17

| Day | 2% Mupirocin | | | Placebo | | | Control | | |
|---|---|---|---|---|---|---|---|---|---|
| 0 | 7.875 | 7.3979 | 7.3979 | 7.875 | 7.3979 | 7.3979 | 7.875 | 7.3979 | 7.3979 |
| 1 | 6.903 | 6.954 | 6.178 | 8.301 | 8.8129 | 8.602 | 8.8129 | 8.8129 | 8.602 |
| 2 | 6.301 | 7.176 | 6 | 8.301 | 8.397 | 8.698 | 8.301 | 8.397 | 8.698 |
| 3 | 5.875 | 5.875 | 6.477 | 8.301 | 8.845 | 8.602 | 8.301 | 8.176 | 8.602 |
| 4 | 5.477 | 5.875 | 6.477 | 8.602 | 8.301 | 8.301 | 8.176 | 8.301 | 8 |
| 5 | 6.477 | 5.875 | 5.875 | 8 | 7.845 | 8 | 8 | 7.903 | 7.845 |

In case of 2% mupirocin on 1^{st} day bacterial load reduces to 1 log CFU and on the final day of experiment it shows 1.5 log CFU reduction. No such log CFU difference was found between control and placebo group.

Table represents the experiment performed in triplicate manner. “0” day represents infection establishment and from day 1 single dose of treatment was started up to day 5. The above table shows the log 10 CFU values for 5 consecutive days.

### Example 16

Effect of the In-Vivo Potency of Cellulose Membranes Impregnated with Mupirocin

In-vivo effect of impregnated cellulose membranes with mupirocin were evaluated by quantitative analysis of the infected wound by estimating the total bacterial load in a 1 cm² skin patch. The bacterial CFU load in the skin patch of control group on zero day was 3.5×10⁷. Cellulose membrane impregnated with Mupirocin (50 μg (0.00018%)/patch) was able to reduce 0.5 log CFU by calculating average mean in three infected mice on the 5^{th} day of treatment. CFU count of 1.5×10⁷ was recovered from untreated group on the 5^{th} day of dissection. No log difference was seen between control and cellulose membrane group which concludes that cellulose membrane alone does not have any antibacterial activity in-vivo (50 μg (0.00018%)/patch) BC group has good efficacy than 0.05% mupirocin.

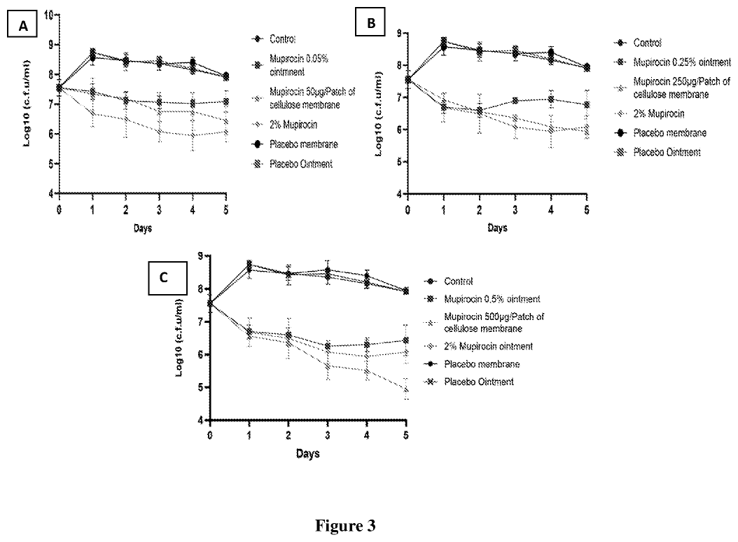

In second in-vivo experiment the bacterial CFU load in the skin patch 24 hr. after establishing infection the CFU count was 3×10⁷. In this study same group of mice were treated with one time application along with Cellulose membranes impregnated with mupirocin at concentration (250 μg (0.0081%)/patch). Results showed 1.5 log C.F.U reduction on 5^{th} day of treatment (FIG. 3). The CFU count of untreated group was 3.0×10⁷ at the end of the day of experiment. 2% mupirocin group reduces bacterial load to 1 log CFU on the first day of treatment. On the 5^{th} day of experiment 1×10⁶ CFU/ml was found. A total of 1.5 log CFU reduction was seen on the final day of treatment. BC mupirocin (250 μg (0.0081%)/patch) group shows good efficacy than 0.25% mupirocin.

In 3^{rd} in-vivo experiment same strategy was followed as performed in previous experiment except the impregnation concentration of mupirocin on the cellulose membrane. 500 μg (0.0162%)/patch of mupirocin impregnated cellulose patches were used in study at single application after 24 hrs. of established infection (FIG. 2). There was a clear 2.5 log CFU reduction in treatment group on 5th day which shows better efficacy than one time application of mupirocin @ 2% alone. BC having mupirocin (500 μg (0.0162%)/patch) shows a very good efficacy than the rest of the group including 2% and 0.5% mupirocin.

### Advantages of the Invention

The present disclosure discloses a method for the cost effective production of bacterial cellulose via fermentation process for transdermal drug delivery. The developed antibacterial bio-cellulosic patches are environment friendly, non-toxic, biocompatible & completely biodegradable. Transdermal delivery is direct-to-bloodstream delivery while bypassing the liver's metabolic activity. The medication is supplied gradually and constantly, rather than in a large, single dose. The patches utilize the skin's natural barrier properties in order to achieve a constant permeation of the drug and achieve steadier blood levels. These patches are painless, eliminating the need for injections that can cause patient irritation and discomfort. Overall, the present disclosure provides a composition for anti-bacterial bio-cellulosic patches/membranes useful for transdermal drug delivery and a process for the preparation thereof.

I/We claim:

1. A composition for antibacterial bio-cellulosic patches comprising:
[a] bacterial cellulose (BC) membrane;
[b] mupirocin in the range of 0.00018% to 0.033% taken from mupirocin stock solution of concentration 50 mg/ml dissolved in methanol; and
[c] glycerol in the range of 1.0% to 3.0%;
wherein [b] and [c] are loaded onto [a].

2. The composition for antibacterial bio-cellulosic patches as claimed in claim 1, wherein 500 μg mupirocin is loaded onto 3100 mg of the bacterial cellulose membrane.

3. The composition for antibacterial bio-cellulosic patches as claimed in claim 1, wherein 0.0162% mupirocin is loaded onto the bacterial cellulose membrane.

4. The composition for antibacterial bio-cellulosic patches as claimed in claim 1, wherein 2.5% glycerol is loaded onto the bacterial cellulose membrane.

5. A process for the preparation of the composition for antibacterial bio-cellulosic patches as claimed in claim 1, wherein the steps comprising:
(a) culturing the isolated bacterial strain of Komagataeibacter hansenii (MBS-8) designated as MTCC 13036 in M5 medium comprising glucose 0.5%, glycerol 4%, peptone 0.5%, yeast extract 0.25%, disodium hydrogen phosphate 0.27%, citric acid 0.015% having pH in the range of 5.5 to 6.2 at a temperature ranging from 28 to 30° C. for a period of 7 to 9 days under static conditions to obtain a bacterial cellulose membrane/bio-cellulosic (BC) patch on the surface of the medium;
(b) The BC membrane obtained in step [a] was washed with boiled 1N NaOH having a temperature in the range of 80 to 90° C. for a period of 1 to 2 hr. and then washed with a weak acid followed by washing with distilled water for 2-3 times until the pH becomes neutral;
(c) the washed BC membrane obtained in step [b] was dipped in distilled water and autoclaved at a temperature of 115 to 120 degree C. for 15 to 20 minutes to obtain a sterilized BC membrane;
(d) the sterilized bacterial cellulose membrane was weighed and then compressed by hands between two acrylic plates for the removal of 50-60% of their water content to obtain a drained BC membrane;
(e) the drained BC membrane obtained in step [d] was soaked in potassium phosphate buffered solution having pH 7.4 containing mupirocin in the range of 50 to 1000 microgram and glycerol in the range of 0.5 to 5.0% for a duration of 24 to 48 hours at room temperature to assure complete absorption of the drug onto the membrane; and
(f) after the drug absorption, the antibacterial bio-cellulosic patches/membranes obtained were dried at temperature ranging from 30 to 40° C. in a ventilated oven for 10 to 16 hours to obtain the desired antibacterial bio-cellulosic patches/membranes.

6. The process as claimed in claim 5, wherein culturing of the isolated bacterial strain MTCC 13036 is done at a temperature of 28° C. for 8 days.

7. The process as claimed in claim 5, wherein the weak acid is glacial acetic acid.
